(19) United States
(12) Patent Application Publication
(10) Pub. No.: US 2009/0117058 A1
(43) Pub. Date: May 7, 2009

(54) Toothpaste composition for tooth whitening

(76) Inventors: Byung-Ryeul Lee, Seoul (KR); Kye-Hong Kang, Kyeongki-do (KR); Dae-Kyeong Kim, Kyeongki-do (KR); Ki-Jung Kim, Kyeongki-do (KR)

Correspondence Address: CLARK & BRODY, 1090 VERMONT AVENUE, NW, SUITE 250, WASHINGTON, DC, 20005, US

(21) Appl. No.: 11/989,283

(22) PCT Filed: Jun. 13, 2006
(86) PCT No.: PCT/KR2006/002257

§ 371 Date: Jan. 24, 2008

(30) Foreign Application Priority Data
Jul. 25, 2005 (KR) 10-2005-0067140

(51) Int. Cl. A61K 8/22; A61Q 11/00

(52) U.S. Cl. 424/53

(57) ABSTRACT

The present invention relates to a toothpaste composition having tooth whitening effect, and more specifically, to a toothpaste composition which contains hydrogen peroxide, as peroxide releasing oxygen free radicals for the whitening effect, and silica with reduced contents of metal ions, to overcome the difficulty in long-term storage due to the release of oxygen free radicals resulting from degradation of peroxide by metal ions released from other components in the composition, and unsatisfactory tooth whitening effect. The toothpaste composition of the present invention does not release oxygen free radicals during storage, and so has excellent preservativeness and sustained tooth whitening effect.

### TECHNICAL FIELD

The present invention relates to a toothpaste composition having excellent tooth whitening effect and preservativeness, and more particularly, to a toothpaste composition for tooth whitening containing hydrogen peroxide, as peroxide which is easily degraded and releases oxygen free radicals upon contact with teeth, and purified silica substantially free from metal ions as an abrasive.

### BACKGROUND ART

Teeth color change is generally classified into endogenous teeth color change due to teeth nerve injury, aging or over-dose of antibiotics such as tetracycline, and exogenous teeth color change due to leftovers deposited on teeth surface, nicotine or tar of tobacco, coffee, tea, etc.

For the whitening and prevention of the color change of teeth, a tooth whitening agent has been known for a long time. Most tooth whitening agents on a market contain hydrogen peroxide or urea peroxide as peroxide to release oxygen free radicals.

It has been well known that peroxides have excellent tooth whitening effect. However, peroxides have problems of poor compatibility with other toothpaste components and low stability for long-term storage.

An abrasive grinds the surface of teeth in a simple physical manner to make it white and sleek. However, repeated grinding results in excessive abrasion of teeth, causing cervical abrasion. Further, commonly used abrasives contain impurities, large amounts of transition metal ions including iron (III) ions. The large amounts of transition metal ions including iron (III) ions act as a catalyst to degrade peroxides including hydrogen peroxide. Therefore, peroxides are rapidly degraded and gas is generated in a toothpaste product, and so toothpastes containing an abrasive have problems of tube-expansion and formulation-separation during storage and distribution.

To overcome the above problems, U.S. Pat. No. 5,085,853 discloses a specialized toothpaste container with a dual compartment dispenser in which a gel containing hydrogen peroxide and a toothpaste formulation containing sodium hydrogen carbonate are held in separate compartments, and intermingled when dispensed. This specialized toothpaste container is designed to hold an oxygen free radical supplier and a metal ion activating the oxygen free radical supplier in isolation form from one another. Utilization of this container, however, is limited because of high cost for subsidiaries and facilities, inconvenience due to increased container volume and difficulties in dual filling of components.

The encapsulation of hydrogen peroxide is an alternative. That is, encapsulated hydrogen peroxide is contained in toothpaste to prevent its reaction with incompatible components therein, and when the capsule is ruptured by physical impact of brushing, the hydrogen peroxide is released from the ruptured capsule. U.S. Pat. No. 4,980,154 discloses a method of applying encapsulated hydrogen peroxide to toothpaste, wherein the capsule has been made from ethyl cellulose and epolene wax known as C-10. This method, however, has problems that the effect of peroxides is decreased unless the coated (encapsulated) peroxide is rapidly degraded in the oral cavity and stability with the lapse of time cannot be ensured since the perfect encapsulation of hydrogen peroxide is nearly impossible under the current technologies.

The present inventors have conducted extensive studies to solve the above-described problems of whitening toothpaste compositions containing peroxide as tooth whitening agent, and to provide a whitening toothpaste composition with improved preservativeness by inhibiting degradation of peroxide to prevent the release of oxygen free radicals. As a result, the present inventors prepared a toothpaste composition for tooth whitening containing only ingredients compatible with peroxide and a purified abrasive. An abrasive is essential ingredient for toothpaste in order to eliminate leftover, dental plaque and contaminants on tooth surface. However, conventional abrasives contain a large amount of metal impurities. So, the toothpaste composition for tooth whitening of the invention contains purified silica, that is, silica purified by a separate purification process, to fundamentally prevent the degradation of peroxide thereby to have improved preservativeness.

### DISCLOSURE

### Technical Problem

It is an object of the present invention to provide a toothpaste composition for tooth whitening with improved preservativeness and stability by employing purified silica.

It is another object of the present invention to provide a toothpaste composition for tooth whitening with improved whitening effect as well as preservativeness by employing a suitable amount of peroxide.

### Technical Solution

To achieve the above objects, the present invention provides a toothpaste composition for tooth whitening, characterized by containing an abrasive purified to contain 30 ppm or less of iron ions as metal impurities, thereby to protect a peroxide from being degraded.

The toothpaste composition of the present invention contains 1 to 9 weight % of peroxide as a whitening agent and 5 to 20 weight % of purified silica as an abrasive based on the total weight of the composition.

Hereinafter, the present invention will be described in detail.

The toothpaste composition of the present invention contains peroxide as an oxygen free radical supplier, the peroxide being one or more selected from hydrogen peroxide, urea peroxide, calcium peroxide, sodium pyrophosphate peroxide or sodium percarbonate, at 0.3-3.5 weights as hydrogen peroxide based on the total weight of the composition. In addition, it contains purified silica as an abrasive at 5-20 weight %.

Hydrogen peroxide, among peroxides acting as a whitening agent in a toothpaste composition for whitening, has a high acidity of pH 1.8-3.5. So, it is stable under an acidic pH, but has oxidation effect more predominant than whitening effect. On the contrary, under a basic pH, the hydrogen peroxide has whitening effect more predominant than oxidation effect, but is likely degraded into water and hydrogen, making its long-term storage difficult. To solve the problem, a stabilizer may be added, and examples of the stabilizer, which can be used for peroxide, include ultra condensed phosphate, polyvinyl pyrrolidone, polyacrylic acid, hydroxyl ethyl cellulose and alkyl carboxylate.

Another means to overcome the above problem is to add a mixture of hydrogen peroxide with another peroxide to a whitening toothpaste composition, thereby to inhibit the degradation of hydrogen peroxide and to improve whitening effect. Particularly, a mixture of hydrogen peroxide and sodium percarbonate at the weight ratio of 1:1-2:1, is efficient for inhibiting degradation of hydrogen peroxide and improving whitening effect.

Purified silica employed in the present invention may be prepared according to any conventional method for eliminating impurities from minerals like inorganic powder. For example, silica can be purified, by eliminating impurities by a chemical method such as acid leaching. The acid leaching is a technology widely used not only to recover useful metals from metallic minerals but also to eliminate rust from the metal surface and irons from nonmetallic minerals. Acids used for the leaching process may be classified into inorganic acids and organic acids, but inorganic acids are preferred because they have higher leaching rate and reaction rate than organic acids, and so inorganic acids are used in most cases. Particularly, silica powder is immersed and stirred in an inorganic acid solution to remove impurities. The solution is washed with purified water to eliminate remaining impurities and the acid solution. The resultant is dried at high temperature for a given time, and then, pulverized by a pulverizer to produce the purified silica abrasive.

The purified silica employed in the present invention contains 50 ppm or less, more preferably 30 ppm or less, of iron as metallic impurities. The particle diameter of the silica powder is 6-11 μm and the particles with the diameter of 53 μm or more are contained at 0.1% or less, providing freshness without sandy feeling during the brushing. If the iron content is higher than 50 ppm, satisfactory effect cannot be obtained.

Other components employed for the toothpaste of the invention are not particularly limited, but it is preferable to use component containing few impurities and highly compatible with peroxide including a wetting agent such as glycerin and polyethylene glycol, and a thickener such as nonionic thickeners, e.g. poloxamer 407, polyvinyl alcohol and polyvinyl pyrrolidone. In addition, additives employed in minor amounts such as sweeteners, pH regulators, preservatives, coloring agents, binders, foaming agents and flavors can be included. Anticaries agents, anticalculus agents and other medicinal components can also be included alone or as a mixture of two or more. For example, as a sweetener, sodium saccharin, aspartame and stevioside can be used. As a pH regulator, phosphoric acid, citric acid, sodium phosphate, trisodium phosphate and sodium citrate can be used. As a preservative, methyl p-hydroxybenzoate, propyl p-hydroxybenzoate and sodium benzoate can be used. As an anticaries agent, sodium fluoride or sodium monofluorophosphate can be used. As an anticalculus agent, sodium pyrophosphate, potassium pyrophosphate, zinc chloride and zinc citrate can be used. As a medicinal component, aminocaproic acid, allantoinhydroxyaluminate, chlorohexidine, tocopherol acetate and pyridoxine hydrochloride can be used alone or as a mixture of two or more thereof. As a foaming agent, anionic and nonionic surfactants such as sodium lauryl sulfate, sodium N-lauroylsarcosylate, sucrose fatty acid ester, polyoxy ethylene hydrogenated caster oil and sorbitan fatty acid ester can be used alone or as a mixture of two or more. As a flavor, peppermint oil, spearmint oil, menthol, anethole and eugenol can be used in mixture.

### ADVANTAGEOUS EFFECT

The toothpaste for tooth whitening according to the present invention employs purified silica for stabilizing peroxides used as a whitening agent, and therefore, can stabilize hydrogen peroxide in the toothpaste formulation, thereby to keep hydrogen peroxide stably without degradation of oxygen free radicals during the usage of toothpaste and to provide excellent tooth whitening effect.

### MODE FOR CARRYING OUT THE INVENTION

The present invention will be more specifically explained with reference to the following examples and comparative examples, but they should not be construed to limit the scope of the present invention in any manner. It will be appreciated that those skilled in the art, on consideration of this disclosure, may make various modifications without departing from the spirit of the present invention, which also fall with the scope of the present invention.

### PREPARATION EXAMPLE 1

### Preparation of Purified Silica

A solution containing 0.5 weight % of hydrochloric acid was prepared, and silica (iron content: 140 ppm) and the solution were mixed at the weight ratio of 1:4. Then, the mixture was stirred for one hour to eliminate metal components contained in the silica. The mixture of silica and HCl solution was centrifuged at 1,500 rpm for 5 minutes to precipitate the silica. The precipitated silica was mixed with purified water at the ratio of 1:1, and the mixture was stirred for 10 minutes to eliminate remaining metal components. Washing with purified water was repeated 3˜4 times. After centrifugation, the precipitated silica was dried at 105° C. for 3 days, followed by pulverizing to give purified silica (iron content: 23 ppm). The purified silica used in the present invention contained 50 ppm or less, more preferably 30 ppm or less, iron as impurities.

To analyze the total iron content, samples were pretreated as follows. 1 g of the sample was introduced in a vessel for microwave digestion, and 4 mL of nitric acid and 4 mL of hydrofluoric acid were added thereto. The mixture was allowed to stand at room temperature until reaction stopped. The vessel was assembled and equipped to the microwave digestion device to perform the degradation of the sample. Upon completion of the degradation of the sample, the sample was allowed to stand at room temperature until it cooled down. After the sample completely cooled down, the sample was transferred to a 50 mL flask while filtering, and ultra pure water was added thereto to a marked line. The resulting solution was used as a test solution.

The iron contents in the sample prepared above and the standard solution were measured using AAS. First, 1,000 mg/kg Fe certified standard solution was diluted to prepare standard solutions of at least three different concentrations. Optical densities of the standard solutions were measured by AAS to produce calibration curve. Optical density of the test solution was measured by AAS to measure Fe content in the test solution.

### PREPARATION EXAMPLE 2

A toothpaste was prepared according to a conventional method using the whitening toothpaste compositions as set forth in the following Table 1. As shown in Table 1, formulations 1-6 contained the purified silica prepared in Preparation Example 1 and comparative formulation 1 contained unpurified silica.

TABLE 1

| | Formu- | Formu- | Formu- | Formu- | Formu- | Formu- | Comparative |
|---|---|---|---|---|---|---|---|
| | lation | lation | lation | lation | lation | lation | formulation |
| Component | 1 | 2 | 3 | 4 | 5 | 6 | 1 |
| Poloxamer | 22 | 22 | 22 | 22 | 22 | 22 | 22 |
| 407 | | | | | | | |
| Glycerin | 10 | 10 | 10 | 10 | 10 | 10 | 10 |
| Sodium | 0.3 | 0.3 | 0.3 | 0.3 | 0.3 | 0.3 | 0.3 |
| stanate | | | | | | | |
| Sodium | 0.1 | 0.1 | 0.1 | 0.1 | 0.1 | 0.1 | 0.1 |
| lauryl | | | | | | | |
| sulfate | | | | | | | |
| Flavor | 0.1 | 0.1 | 0.1 | 0.1 | 0.1 | 0.1 | 0.1 |
| Sodium | 0.2 | 0.2 | 0.2 | 0.2 | 0.2 | 0.2 | 0.2 |
| saccharin | | | | | | | |
| Hydrogen | 2.15 | 2.15 | 2.15 | 2.15 | 2.15 | 2.15 | 2.15 |
| peroxide | | | | | | | |
| (35%) | | | | | | | |
| Sodium | — | — | — | — | — | 0.50 | — |
| percarbonate | | | | | | | |
| Unpurified | — | — | — | — | — | — | 10 |
| silica | | | | | | | |
| (140 ppm Fe) | | | | | | | |
| Purified | 10 | — | 5 | 20 | 30 | 10 | — |
| silica | | | | | | | |
| (23 ppm Fe) | | | | | | | |
| Phosphoric | 0.2 | 0.2 | 0.2 | 0.2 | 0.2 | 0.2 | 0.2 |
| acid | | | | | | | |
| Purified | ad 100 | ad 100 | ad 100 | ad 100 | ad 100 | ad 100 | ad 100 |
| water | | | | | | | |

### EXPERIMENTAL EXAMPLE 1

### Evaluation of Product Stability

To compare preservativeness of each composition listed in Table 1, remaining amount of hydrogen peroxide was measured to determine the stability of oxygen free radicals, and the state of the product retained in tube was observed. The results are shown in Table 2.

Test Method

2 g of the sample was accurately taken and dissolved in 50 mL of 0.25 mol/L sulfuric acid. Then, 10 mL of isopropanol was added thereto to make a mixture. The mixture was titrated with 0.1 mol/L Ce(SO₄)₂·2(NH₄)₂SO₄ solution to pale yellow to obtain reduced amounts of remaining hydrogen peroxide (H₂O₂: 34.01) or hydrogen peroxide of other peroxides.

0.1 mol/L Ce(SO₄)₂·2(NH₄)₂SO₄ solution 1 mL=1.701 mg H₂O₂

TABLE 2

| | | Formu- | Formu- | Formu- | Formu- | Formu- | Formu- | Comparative |
|---|---|---|---|---|---|---|---|---|
| | Temperature | lation | lation | lation | lation | lation | lation | formulation |
| | (° C.) | 1 | 2 | 3 | 4 | 5 | 6 | 1 |
| Remain- | Immediately | 100 | 100 | 100 | 100 | 100 | 100 | 100 |
| ing | after | | | | | | | |
| H₂O₂ % | preparation | | | | | | | |
| | 55° C. | 99 | 100 | 100 | 99 | 99 | 99 | 52 |
| | 1 week later | | | | | | | |
| | 55° C. | 99 | 99 | 99 | 99 | 98 | 98 | 26 |
| | 3 week later | | | | | | | |
| | 55° C. | 99 | 99 | 99 | 98 | 98 | 96 | — |
| | 5 week later | | | | | | | |
| Composi- | Immediately | Good | Good | Good | Good | Good | Good | Good |
| tion | after | | | | | | | |
| retained | preparation | | | | | | | |
| in tube | 55° C. | Good | Good | Good | Good | Good | Good | Expansion |
| | 1 week later | | | | | | | |
| | 55° C. | Good | Good | Good | Good | Good | Good | Expansion |
| | 3 week later | | | | | | | and burst |
| | 55° C. | Good | Good | Good | Good | Good | Good | — |
| | 5 week later | | | | | | | |

As shown in Table 2, toothpastes of formulations 1-6 containing the purified silica were proved to have better stability than that of comparative formulation 1.

### EXPERIMENTAL EXAMPLE 2

### Organoleptic Test on Abrasiveness

Organoleptic test was performed to investigate the abrasiveness of toothpastes of formulations 1-6 and comparative formulation 1.

Particularly, toothpastes of formulations 1-6 and comparative formulation 1 were applied to toothbrushes wet with sputum at 1 g per toothbrush, which were given to 30 testees. The testees were let to brush their teeth for 3 minutes. After rinsing with water five times, they were let to evaluate abrasiveness according to 5-point scale. The results of the organoleptic test on abrasiveness are shown in Table 3.

TABLE 3

| Index of | | | | | | | |
|---|---|---|---|---|---|---|---|
| abrasiveness | Formu- | Formu- | Formu- | Formu- | Formu- | Formu- | Comparative |
| (5-point | lation | lation | lation | lation | lation | lation | formulation |
| scale | 1 | 2 | 3 | 4 | 5 | 6 | 1 |
| After | 2.9 | 1.1 | 2.3 | 3.4 | 4.8 | 2.8 | 2.8 |
| brushing | | | | | | | |

Evaluation basis: Very strong 5, Strong 4, Medium 3, Weak 2, Very weak 1

As shown in Table 3, the toothpaste of formulation 2 containing no abrasive exhibited weak abrasiveness, while the toothpaste of formulation 5 containing 30 weights of the purified silica as an abrasive showed a very strong abrasiveness. Therefore, the preferable content of abrasive was concluded to be 5-20 weight %.

### EXPERIMENTAL EXAMPLE 3

### Measurement of In Vivo Tooth Whitening Effect

To investigate the tooth whitening effect of the toothpastes according to the present invention, 150 healthy men and women were selected as a sample to measure teeth color before and after using the toothpaste.

The original tooth colors of testees were measured by SHADEEYE-EX (SHOFU Co. LTD., Japan). SHADEEYE-EX tip was vertically set to the teeth surface in the middle of mesio-distal diameter, 2 mm above the buccocervical region of central incisor at the left of upper jaw, and the surface of teeth was irradiated with care not to shine out, to measure and collect baseline values (primary standard value). Then, the testees were let to brush their teeth with the toothpastes of formulations 1-6 and comparative formulation 1 three times a day for 2 months. During the experiment, food was taken according to each one's dietary habit, but on the day of measuring tooth color, coffee, cola and tobacco were prohibited. Chromaticity was measured (secondary measurement) after two-month use of the toothpastes of formulations 1-6 and comparative formulation 1. The results are shown in Tables 4 and 5.

TABLE 4
Tooth color changes after two-month use
of the toothpastes of the invention

| | Baseline mean value | Mean value after 2 months |
|---|---|---|
| Comparative | 7.90 | 7.32 |
| formulation 1 | | |
| Formulation 1 | 7.89 | 6.15 |
| Formulation 6 | 7.87 | 6.14 |

TABLE 5
Tooth whitening effect after two-month
use of the toothpastes of the invention

| | Better | Same | Worse |
|---|---|---|---|
| Comparative | 18 (36%) | 32 (64%) | 0 (0%) |
| formulation 1 | | | |
| Formulation 1 | 34 (68%) | 16 (32%) | 0 (0%) |
| Formulation 6 | 35 (70%) | 15 (30%) | 0 (0%) |

As shown in Tables 4 and 5, the toothpastes of formulations 1 and 6 exhibited excellent tooth whitening effect, compared with that of comparative formulation 1, because hydrogen peroxide in the formulations 1 and 6 was more stable.

### INDUSTRIAL APPLICABILITY

The present invention provides a toothpaste composition for tooth whitening containing hydrogen peroxide, as peroxide which is easily degraded and releases oxygen free radicals upon contact with teeth, and purified silica substantially free from metal ions as an abrasive, thereby to have excellent tooth whitening effect and preservativeness.

1. A whitening toothpaste composition with improved preservativeness, characterized by containing peroxide and purified silica.

2. The composition according to claim 1, wherein the peroxide is one or more selected from the group consisting of hydrogen peroxide, urea peroxide, calcium peroxide, sodium pyrophosphate peroxide and sodium percarbonate.

3. The composition according to claim 2, wherein the peroxide is contained at 0.3-3.5 weight % based on the total weight of the composition.

4. The composition according to claim 2, wherein the purified silica is contained at 5-20 weight % based on the total weight of the composition.

5. The composition according to claim 4, wherein the purified silica contains 50 ppm or less of iron (III).

6. The composition according to claim 5, wherein the purified silica is prepared by the process comprising the steps of: a) mixing silica and 0.5 weight % HCl solution at the weight ratio of 1:2-1:6 with stirring, and centrifuging the mixture; b) mixing the silica precipitated in step a) and purified water at the weight ratio of 1:0.5-1:8 with stirring, and washing the mixture; and c) drying and pulverizing the silica precipitated in step b) to give purified silica.

7. The composition according to claim 6, which contains one or more components selected from the group consisting of wetting agents, thickeners, sweeteners, pH regulators, preservatives, coloring agents, binders, foaming agents, flavors, anticaries agents, anticalculus agents and medicinal ingredients.

8. The composition according to claim 7, characterized by containing a hydrogen peroxide stabilizer selected from the group consisting of ultra condensed phosphate, polyvinyl pyrrolidone, polyacrylic acid, hydroxyl ethyl cellulose and alkyl carboxylate.

9. The toothpaste composition according to claim 8, wherein the purified silica has an average particle diameter of 6-11 μm, and has 0.1% or less of particles with 53 μm or more of maximum particle diameter.

10. The composition according to claim 9, wherein the hydrogen peroxide and sodium percarbonate are mixed at the weight ratio of 1:1-2:1 to improve tooth whitening effect.
